(19) United States
(12) Patent Application Publication
(10) Pub. No.: US 2022/0257148 A1
(43) Pub. Date: Aug. 18, 2022

(54) FACTOR ESTIMATION SYSTEM AND FACTOR ESTIMATION METHOD

(71) Applicant: Panasonic Intellectual Property Management Co., Ltd., Osaka (JP)

(72) Inventors: Takahiro AIHARA, Osaka (JP); Kengo WADA, Osaka (JP); Taichi HAMATSUKA, Osaka (JP); Yoshihiro MATSUMURA, Osaka (JP); Yoshikuni SATO, Tokyo (JP); Takahiro HIYAMA, Tokyo (JP)

(21) Appl. No.: 17/632,781

(22) PCT Filed: Jul. 29, 2020
(86) PCT No.: PCT/JP2020/029157

§ 371 Date: Feb. 3, 2022

(30) Foreign Application Priority Data
Sep. 13, 2019 (JP) 2019-167171

(51) Int. Cl. A61B 5/11; G06V 40/20; G06V 20/52; A61B 5/00

(52) U.S. Cl. CPC A61B 5/1117; G06V 40/25; G06V 20/52; A61B 5/7278; A61B 5/7275; A61B 5/112; A61B 5/1128

(57) ABSTRACT

A factor estimation system is a factor estimation system that estimates a factor of fall risk indicating a possibility of a fall of a measured person, including: a calculator that obtains body motion data indicating body motion of the measured person during walking, and calculates two or more walking parameters of the measured person based on the body motion data obtained; and a factor analyzer that estimates, based on the two or more walking parameters, one or more main components that are included in the factor of the fall risk of the measured person and are based on the two or more walking parameters, and outputs an estimation result.

### TECHNICAL FIELD

The present disclosure relates to a factor estimation system and a factor estimation method that estimate a factor of fall risk indicating the possibility of a fall of a person to be measured (hereafter “measured person”).

### BACKGROUND ART

Methods of evaluating or determining fall risk are conventionally proposed (for example, see Patent Literature (PTL) 1). PTL 1 discloses a method of evaluating the fall risk of a measured person based on the number of one-foot taps as an index of motor function and a timed up to go (TUG) test value as an index of musculoskeletal ambulation disability symptom complex.

### CITATION LIST

### Patent Literature

[PTL 1]

Japanese Unexamined Patent Application Publication No. 2017-042618

### SUMMARY OF INVENTION

### Technical Problem

With the method described in PTL 1, the fall risk can be evaluated. However, in the case where the measured person has fall risk, the factor of the fall risk is unknown. Hence, a guardian (for example, a caregiver) of the measured person who has fall risk may be unable to appropriately make a suggestion for reducing the fall risk to the measured person.

The present disclosure accordingly has an object of providing a factor estimation system and a factor estimation method that can estimate a factor of fall risk.

### Solution to Problem

A factor estimation system according to an aspect of the present disclosure is a factor estimation system that estimates a factor of fall risk indicating a possibility of a fall of a measured person, the factor estimation system including: a calculator that obtains body motion data indicating body motion of the measured person during walking, and calculates two or more walking parameters of the measured person based on the body motion data obtained; and an estimator that estimates, based on the two or more walking parameters, one or more main components that are included in the factor of the fall risk of the measured person and are based on the two or more walking parameters, and outputs an estimation result.

A factor estimation method according to an aspect of the present disclosure is a factor estimation method of estimating a factor of fall risk indicating a possibility of a fall of a measured person, the factor estimation method including: obtaining body motion data indicating body motion of the measured person during walking; calculating two or more walking parameters of the measured person based on the body motion data obtained; and estimating, based on the two or more walking parameters, one or more main components that are included in the factor of the fall risk of the measured person and are based on the two or more walking parameters, and outputting an estimation result.

### Advantageous Effects of Invention

The factor estimation system, etc. according to an aspect of the present disclosure can estimate a factor of fall risk.

### BRIEF DESCRIPTION OF DRAWINGS

FIG. 1 is a diagram illustrating a schematic structure of a factor estimation system according to Embodiment 1.

FIG. 2 is a block diagram illustrating a functional structure of the factor estimation system according to Embodiment 1.

FIG. 3 is a diagram illustrating an example of a formula for calculating a fall risk value by a risk analyzer according to Embodiment 1,

FIG. 4 is a flowchart illustrating operation performed before estimation operation in the factor estimation system according to Embodiment 1.

FIG. 5 is a diagram illustrating an example of first correspondence information.

FIG. 6 is a diagram illustrating an example of second correspondence information.

FIG. 7 is a flowchart illustrating estimation operation of estimating a factor of fall risk in the factor estimation system according to Embodiment 1.

FIG. 8 is a block diagram illustrating a functional structure of a factor estimation system according to Embodiment 2.

FIG. 9 is a flowchart illustrating operation in the factor estimation system according to Embodiment 2.

FIG. 10 is a diagram illustrating an example of the correspondence relationship between factors and intervention methods.

FIG. 11 is a diagram illustrating the vertical displacement of the body of a measured person during walking.

FIG. 12 is a diagram illustrating a frequency analysis result in the case where the cognitive function of the measured person is normal.

FIG. 13 is a diagram illustrating a frequency analysis result in the case where the cognitive function of the measured person is low.

FIG. 14 is a block diagram illustrating a functional structure of a factor estimation system according to Embodiment 3.

FIG. 15 is a flowchart illustrating operation in the factor estimation system according to Embodiment 3.

FIG. 16A is a flowchart illustrating an example of operation in a risk determiner according to Embodiment 3,

FIG. 16B is a flowchart illustrating another example of operation in the risk determiner according to Embodiment 3.

### DESCRIPTION OF EMBODIMENTS

Embodiments will be described below, with reference to the drawings. The embodiments described below each show a general or specific example. The numerical values, shapes, materials, structural elements, the arrangement and connection of the structural elements, steps, the order of steps, etc. shown in the following embodiments are mere examples, and do not limit the scope of the present disclosure. Of the structural elements in the embodiments described below, the structural elements not recited in any one of the independent claims are described as optional structural elements.

Each drawing is a schematic and does not necessarily provide precise depiction. The substantially same structural elements are given the same reference signs throughout the drawings, and repeated description may be omitted or simplified.

In the specification, the terms indicating the relationships between elements, such as “equal”, the numerical values, and the numerical ranges are not expressions of strict meanings only, but are expressions of meanings including substantially equivalent ranges, for example, allowing for a difference of about several percent.

### Embodiment 1

### [1-1. Schematic Structure of Factor Estimation System]

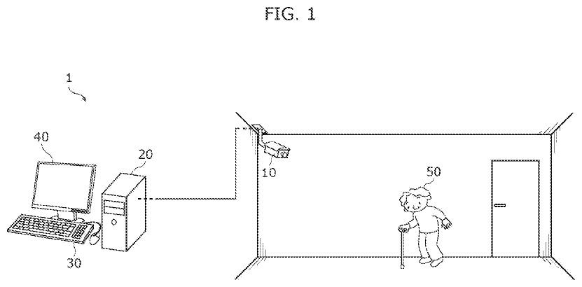

FIG. 1 is a diagram illustrating a schematic structure of factor estimation system 1 according to this embodiment. As illustrated in FIG. 1, factor estimation system 1 includes measurement device 10, estimation device 20, input device 30, and display device 40.

Factor estimation system 1 measures the body motion of measured person (i.e. person to be measured) 50 during walking (i.e. during gait) by measurement device 10 (for example, camera), to generate moving image data. Measurement device 10 is installed, for example, at the ceiling or wall of a nursing home or a nursing facility, and constantly captures an image of the room interior. Estimation device 20 analyzes the walking state of measured person 50 based on the moving image data captured (generated) by measurement device 10, and estimates a factor of fall risk of measured person 50. The estimation result is displayed on display device 40. The moving image data is an example of body motion data. Measured person 50 is an example of a subject.

Factor estimation system 1 using such measurement device 10 can evaluate the past and current estimation results of measured person 50, by accumulating moving image data constantly captured by measurement device 10. Factor estimation system 1 can also estimate the factor of fall risk of measured person 50, without being noticed by measured person 50. Measurement device 10 is not limited to constantly capturing an image of measured person 50.

### [1-2. Functional Structure of Factor Estimation System]

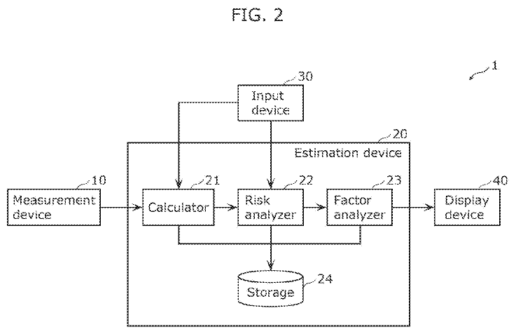

A functional structure of factor estimation system 1 according to this embodiment will be described below, with reference to FIG. 2. FIG. 2 is a block diagram illustrating the functional structure of factor estimation system 1 according to this embodiment. Factor estimation system 1 is a system that promptly estimates the factor of fall risk of measured person 50 by measuring the body motion of measured person 50 during walking.

As illustrated in FIG. 2, factor estimation system 1 includes measurement device 10, estimation device 20, input device 30, and display device 40.

Measurement device 10 is a device that measures the body motion of measured person 50 during walking. In this embodiment, measurement device 10 is a camera for capturing moving image data of measured person 50 during walking. Measurement device 10 may be a camera using a complementary metal oxide semiconductor (CMOS) image sensor, or a camera using a charge coupled device (CCD) image sensor.

The framerate (the numbers of frames of image data included in moving image data per second) is not limited, and may be, for example, 40 fps (frames per second) or 60 fps.

Estimation device 20 analyzes the walking state of measured person 50 based on the moving image data captured by measurement device 10, estimates the factor of fall risk of measured person 50, and outputs the estimation result to display device 40. Thus, estimation device 20 can notify, for example, a caregiver who cares for measured person 50 of the estimation result of the factor of fall risk of measured person 50. This enables the caregiver to make a more appropriate suggestion (intervention) for reducing the fall risk to measured person 50. For example, in the case where the caregiver is unaware that measured person 50 has fall risk, factor estimation system 1 can make the caregiver aware that measured person 50 has fall risk by notifying the caregiver of the factor of fall risk. Moreover, in the case where measured person 50 is unaware of having fall risk, factor estimation system 1 can make measured person 50 aware of having fall risk by notifying measured person 50 of the factor of fall risk.

Estimation device 20 includes calculator 21, risk analyzer 22, factor analyzer 23, and storage 24.

Calculator 21 obtains a measurement result (for example, moving image data) from measurement device 10, and calculates walking parameters from the obtained measurement result. For example, calculator 21 obtains moving image data captured by measurement device 10, as body motion data indicating the body motion of measured person 50 during walking. The method of calculating the walking parameters from the moving image data is not limited. For example, the walking parameters may be calculated by image analysis of the moving image data.

It is known that a person suffering from decline in at least one of muscle strength, muscle mass, sense of balance, or cognitive function differs in body motion during walking from a person (healthy person) without decline in at least one of muscle strength, muscle mass, sense of balance, or cognitive function. Therefore, the walking parameters include walking speed, step length, joint angle, and/or lumbar or head displacement that correlate with at least one of muscle strength, muscle mass, sense of balance, or cognitive function. The walking parameters include at least two of walking speed, step length, joint angle, or lumbar or head displacement. An example of the joint angle is the angle of the knee joint.

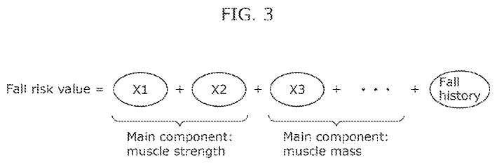

Risk analyzer 22 analyzes the fall risk of measured person 50 based on the walking parameters. For example, risk analyzer 22 analyzes the fall risk of measured person 50 by calculating a fall risk value based on a calculation formula illustrated in FIG. 3. Risk analyzer 22 is an example of a second determiner.

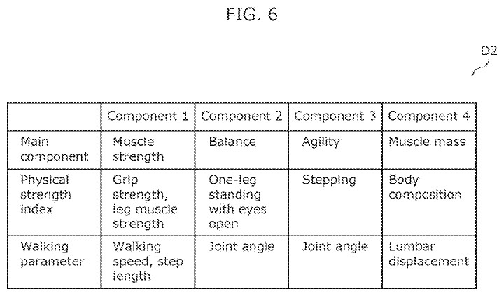

FIG. 3 is a diagram illustrating an example of the formula for calculating a fall risk value by risk analyzer 22 according to this embodiment. Scores X1, X2, and X3 illustrated in FIG. 3 are numeric values based on walking parameters. For example, score X1 is a numeric value based on step length, score X2 is a numeric value based on walking speed, and score X3 is a numeric value based on lumbar position. Each score may be a numeric value based on two or more walking parameters. For example, score X1 may be a numeric value based on step length and walking speed. Although only “muscle strength” and “muscle mass” from among the below-described main components (see FIG. 6) are illustrated in FIG. 3, any other main component(s) may be included. That is, the fall risk value may be calculated based on two or more main components from among the below-described plurality of main components. For example, the fall risk value may be calculated based on all of the below-described plurality of main components.

As illustrated in FIG. 3, risk analyzer 22 adds scores X1, X2, and X3 and a fall history-related score to calculate the fall risk value. Scores X1 and X2 are, for example, each a numeric value based on a walking parameter corresponding to muscle strength. Score X1 may be a numeric value based on walking speed, and score X2 may be a numeric value based on step length. There is a correlation between muscle strength and each of walking speed and step length (see FIG. 6). Score X3 is, for example, a numeric value based on a walking parameter corresponding to balance system (for example, sense of balance). Score X3 may be a numeric value based on lumbar displacement. There is a correlation between muscle mass and lumbar displacement (see FIG. 6).

The fall history-related score is, for example, a numeric value based on whether measured person 50 has fallen and/or the number of falls. As a result of the fall risk value including the fall history-related score, the fall risk can be determined appropriately even in the case where the muscle strength, the muscle mass, etc. are normal. For example, risk analyzer 22 obtains information about fall history via input device 30. Alternatively, risk analyzer 22 may obtain information about fall history by reading the information from storage 24.

Risk analyzer 22 outputs an analysis result corresponding to the fall risk value calculated using the formula illustrated in FIG. 3, For example, risk analyzer 22 may output whether measured person 50 has fall risk and/or output a fall risk level (for example, “high”, “medium”, “low”). Fall risk levels are not limited as long as the number of levels is three or more. Risk analyzer 22 may output the fall risk value.

The formula illustrated in FIG. 3 is an example, and the fall risk value may be calculated using a formula other than the formula illustrated in FIG. 3 as long as the fall risk value is based on the walking parameters. For example, the fall risk value may be calculated by assigning predetermined weights to scores X1 to X3, etc. For example, the fall risk value may be calculated using at least one of addition, subtraction, multiplication, or division. The fall risk value may be calculated further using a numeric value relating to cognitive function. That is, the fall risk may be analyzed further based on the cognitive function of measured person 50.

Referring back to FIG. 2, factor analyzer 23 analyzes the factor of fall risk indicating the possibility of a fall of measured person 50, based on the walking parameters. For example, factor analyzer 23 analyzes the factor of fall risk of measured person 50 based on the walking parameters and correspondence information indicating the correspondence relationship between human physical strength indexes and walking parameters. The physical strength indexes each indicate human physical strength or exercise capacity, and include an item measured in physical strength measurement or the like. For example, the physical strength indexes include grip strength, leg muscle strength, one-leg standing with eyes open, and stepping (for example, side to side jumping). The physical strength indexes may include body composition estimated from a measurement result of a body composition analyzer. The body composition is, for example, a measurement result of a body composition analyzer using bioelectrical impedance analysis (BIA). The walking parameters are not included in the physical strength indexes. The factor includes a main factor (component) influencing human's fall risk. Factor analyzer 23 is an example of an estimator.

Storage 24 is a storage device that stores various data obtained or calculated by each processing unit. For example, storage 24 may store the moving image data obtained from measurement device 10, and the walking parameters calculated by calculator 21. For example, storage 24 may store the analysis results of risk analyzer 22 and factor analyzer 23.

As an example, to analyze the long-term changes of the body motion of measured person 50 during walking, calculator 21 may store the moving image data or calculated walking parameters of measured person 50 in storage 24. As another example, to analyze the long-term changes of the analysis result for measured person 50, risk analyzer 22 or factor analyzer 23 may store the analysis result in storage 24. The long-term period is not limited, and may be, for example, one week, one month, or one year. Hereafter, the moving image data, the walking parameters, and the analysis result are also referred to as information based on body motion.

Thus, estimation device 20 can determine, for example, the current fall risk using information (for example, walking parameters) based on the past body motion of measured person 50 during walking. For example, estimation device 20 can determine whether measured person 50 currently has fall risk.

Storage 24 also stores, for example, a program for each processing unit to carry out a factor estimation method according to the embodiment, and information and data used in factor analysis. Storage 24 is implemented by semiconductor memory, a hard disk drive (HDD), or the like.

Risk analyzer 22 may be omitted from estimation device 20. Estimation device 20 may have any structure that can estimate the factor of fall risk of measured person 50.

The processing units in estimation device 20 may be implemented by one processor, microcomputer, or dedicated circuit having their functions, or implemented by a combination of two or more processors, microcomputers, or dedicated circuits. Calculator 21 and factor analyzer 23 may include a communication module (communication circuit) for performing wire communication or wireless communication. In this case, calculator 21 can use any communication method (communication standard, communication protocol) that enables communication with measurement device 10. Factor analyzer 23 can use any communication method (communication standard, communication protocol) that enables communication with display device 40. Thus, calculator 21 may function as an obtainer, and factor analyzer 23 may function as an outputter.

Estimation device 20 is, for example, a personal computer. Alternatively, estimation device 20 may be a server device. Estimation device 20 may be installed in a building in which measurement device 10 is installed, or installed outside the building.

Input device 30 is a user interface that receives input of certain information from measured person 50. For example, input device 30 receives input of information about the fall history of the measured person. Input device 30 is implemented by hardware keys (hardware buttons), slide switches, a touch panel, and the like.

Display device 40 displays an image based on the analysis result of the factor of fall risk output from estimation device 20. Specifically, display device 40 is a monitor device composed of a liquid crystal panel, an organic EL panel, or the like. As display device 40, an information terminal such as a television, a smartphone, a tablet terminal, or a wearable terminal may be used. Communication between estimation device 20 and display device 40 is, for example, wire communication, but may be wireless communication in the case where display device 40 is a smartphone, a tablet terminal, or a wearable terminal.

### [1-3. Operation in Factor Estimation System]

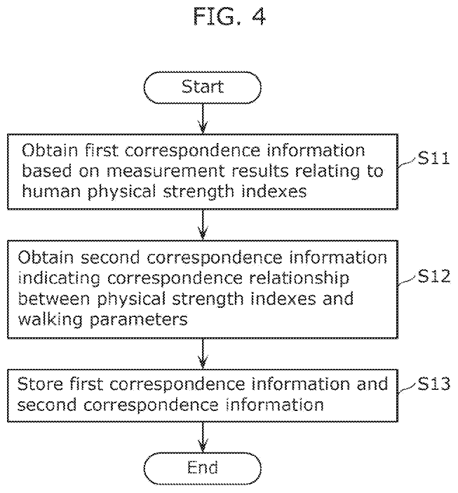

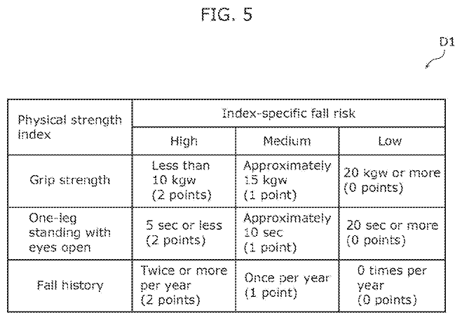

Operation in factor estimation system 1 according to this embodiment will be described below, with reference to FIG. 4 and FIG. 5. FIG. 4 is a flowchart illustrating operation performed before estimation operation in factor estimation system 1 according to this embodiment. Specifically, FIG. 4 illustrates operation performed before analyzing the factor of fall risk of measured person 50.

As illustrated in FIG. 4, calculator 21 obtains first correspondence information based on measurement results relating to human physical strength indexes (S11). For example, calculator 21 obtains the first correspondence information indicating the correspondence relationship between fall risk for each physical strength index and measurement results relating to the physical strength index. FIG. 5 is a diagram illustrating an example of first correspondence information D1. Hereafter, fall risk for each physical strength index is also referred to as index-specific fall risk. The first correspondence information is an example of information indicating a relationship between a human physical strength index and fall risk.

As illustrated in FIG. 5, first correspondence information D1 illustrates the correspondence relationship between physical strength indexes including “grip strength”, “one-leg standing with eyes open”, and “fall history” and index-specific fall risk levels including “high”, “medium”, and “low”. Regarding “grip strength”, the index-specific fall risk is “high” when the grip strength is less than 10 kgw, “medium” when the grip strength is approximately 15 kgw, and “low” when the grip strength is 20 kgw or more. The items and numeric values illustrated in FIG. 5 are an example, and the first correspondence information is not limited to such. The term “approximately” in FIG. 5 denotes a numeric value range of 15 kgw and around 15 kgw in the case of grip strength. The expression “approximately 15 kgw” may denote a numeric value range between the grip strength corresponding to index-specific fall risk “high” and the grip strength corresponding to index-specific fall risk “low”. For example, “approximately 15 kgw” may denote 10 kgw or more and less than 20 kgw.

A score is assigned to each of index-specific fall risk levels “high”, “medium”, and “low”. For example, index-specific fall risk “high” is assigned 2 points, “medium” is assigned 1 point, and “low” is assigned 0 points, although the score assignment is not limited to such. Calculator 21 may obtain a threshold for determining whether measured person 50 has fall risk, based on the score of index-specific fall risk. For example, calculator 21 may obtain a threshold for an arithmetic operation value obtained as a result of arithmetic operation on the scores of the physical strength indexes. The arithmetic operation is, for example, addition, but may be at least one of subtraction, multiplication, or division. The arithmetic operation may be weighted addition or the like. The following will describe an example in which the arithmetic operation is addition and the arithmetic operation value is the sum of the scores of the physical strength indexes. For example, calculator 21 obtains 6 points as a first threshold for determining whether the fall risk of measured person 50 is “high”, and obtains 2 points as a second threshold for determining whether the fall risk of the user is “medium”. The first threshold and the second threshold may be stored, for example, in storage 24.

Calculator 21 then obtains second correspondence information D2 indicating the correspondence relationship between physical strength indexes and walking parameters (S12). For example, calculator 21 may obtain second correspondence information D2 via input device 30. FIG. 6 is a diagram illustrating an example of second correspondence information D2. The second correspondence information is an example of information indicating a relationship between a human physical strength index and two or more walking parameters.

As illustrated in FIG. 6, second correspondence information D2 is information associating components 1 to 4 included in factors of fall risk with main components, physical strength indexes, and walking parameters. The main components each indicate a physical element related to human's fall risk, and are set beforehand. For example, the main components include “muscle strength”, “balance”, “agility”, and “muscle mass”. Regarding component 1 as an example, the physical strength indexes corresponding to the main component “muscle strength” are “grip strength” and “leg muscle strength”, and the walking parameters corresponding to “muscle strength” are “walking speed” and “step length”. In other words, second correspondence information D2 indicates that “walking speed” and “step length” can be used instead of “grip strength” and “leg muscle strength” when estimating the factor of fall risk.

The main component “muscle strength” denotes that one of the factors of a person falling is the muscle strength of the person. The physical strength indexes “grip strength” and “leg muscle strength” are indexes indicating the state of the main component “muscle strength”. The walking parameters correlated with the physical strength indexes “grip strength” and “leg muscle strength” are “walking speed” and “step length”.

The correlation herein may include, in the case where the physical strength index is “grip strength” and the walking parameter is “walking speed”, the correlation between the value of the grip strength and the value of the walking parameter. For example, the correlation may include the correlation that a walking speed of 2 km/h corresponds to a grip strength of 10 kgw.

The correlation between physical strength indexes and walking parameters can be obtained, for example, by regression analysis on the measurement results of the physical strength indexes and walking parameters of a plurality of persons, although the correlation obtainment method is not limited to such.

In component 2, “joint angle” includes, for example, the difference between the joint angles of the left and right legs. Herein, “joint angle” is the angle of a joint relating to walking, such as the angle of the knee joint. For example, the difference between the joint angles of the left and right legs is the difference between the angle of the knee joint of the left leg and the angle of the knee joint of the right leg.

In component 3, “joint angle” includes, for example, the magnitude of the joint angle. Herein, “joint angle” is the angle of a joint relating to walking, such as the magnitude of the angle of the knee joint.

In component 4, “lumbar displacement” includes a displacement of the lumbar. The walking parameter in component 4 is any one or more walking parameters correlated with the value of a body composition analyzer. For example, “head displacement” may be included as a walking parameter instead of or in addition to “lumbar displacement”.

Referring back to FIG. 4, calculator 21 stores first correspondence information D1 and second correspondence information D2 in storage 24 (S13).

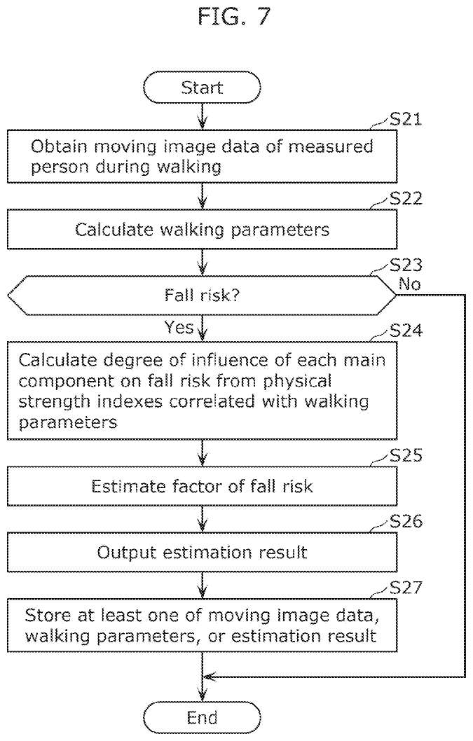

Operation of estimating a factor of fall risk in factor estimation system 1 will be described below, with reference to FIG. 7. FIG. 7 is a flowchart illustrating estimation operation of estimating a factor of fall risk in factor estimation system 1 according to this embodiment.

As illustrated in FIG. 7, calculator 21 obtains moving image data of measured person 50 during walking, from measurement device 10 (S21). The moving image data may be data obtained by capturing an image of measured person 50 walking as usual, or data obtained by capturing an image of measured person 50 walking in a predetermined location in order to estimate the factor of fall risk. For example, the predetermined location may be a passage including a walking surface marked with a marker. The moving image data may be moving image data obtained by capturing an image of measured person 50 from a plurality of points of view.

Following this, calculator 21 calculates walking parameters of measured person 50 based on the moving image data (S22). The method of calculating the walking parameters by calculator 21 is not limited. For example, the walking parameters may be calculated by image analysis of the moving image data. Calculator 21 may, for example, calculate feature points of measured person 50 from the image data, and calculate the walking parameters based on the movement locus of each feature point. In the case where calculator 21 obtains the moving image data of measured person 50 walking along the passage mentioned above, calculator 21 may calculate the feature points by a background differencing technique. Calculator 21 outputs the walking parameters to risk analyzer 22.

Next, risk analyzer 22 determines whether measured person 50 has fall risk, based on the walking parameters (S23). For example, risk analyzer 22 calculates a score for each walking parameter, and, based on the calculated plurality of scores, determines whether measured person 50 has fall risk. Risk analyzer 22 calculates, for example, the score for each walking parameter based on first correspondence information D1 and second correspondence information D2 stored in storage 24. In the case where a walking parameter is walking speed and the walking speed is 2 km/h, for example, risk analyzer 22 recognizes that a walking speed of 2 km/h corresponds to a grip strength of 10 kgw. Risk analyzer 22 then obtains 2 points as the score of the walking speed of 2 km/h, based on the first correspondence information.

For example, risk analyzer 22 calculates the score for each walking parameter and adds the calculated plurality of scores to calculate a fall risk value, as indicated by the formula in FIG. 3. For example, in the case where the sum of the plurality of scores, i.e. the fall risk value, is greater than or equal to a threshold, risk analyzer 22 determines that measured person 50 has fall risk. The threshold in this case is a numeric value for determining whether there is fall risk. The threshold may be a fixed value, or be set for each measured person 50.

For example, in the case where the first threshold (for example, 6 points) and the second threshold (for example, 2 points) are set as thresholds, risk analyzer 22 can determine the degree of fall risk. For example, in the case where the degree of fall risk is greater than or equal to a predetermined degree (for example, greater than or equal to “medium”), risk analyzer 22 may determine that measured person 50 has fall risk.

The method of determining whether measured person 50 has fall risk by risk analyzer 22 is not limited to the above. For example, in the case where the walking speed is less than or equal to a threshold, risk analyzer 22 may determine that measured person 50 has fall risk. Thus, risk analyzer 22 may determine whether measured person 50 has fall risk based on the numeric value of the walking parameter.

Risk analyzer 22 outputs the determination result to factor analyzer 23. Risk analyzer 22 may also store the determination result in storage 24. The determination result output from risk analyzer 22 is an example of a second determination result.

In the case where factor analyzer 23 obtains a determination result indicating that measured person 50 has fall risk from risk analyzer 22 (S23: Yes), factor analyzer 23 calculates, from the physical strength indexes correlated with the walking parameters, the degree of influence of each main component on the fall risk (S24). For example, factor analyzer 23 recognizes that the walking parameters “walking speed” and “step length” correlate with the main component “muscle strength”, based on second correspondence information D2. Factor analyzer 23 calculates the degree of influence of the main component “muscle strength” on the fall risk, based on walking speed and step length. For example, factor analyzer 23 may calculate the degree of influence on the fall risk based on the score of walking speed and the score of step length. Factor analyzer 23 calculates, for example, the sum of the score of walking speed and the score of step length as the degree of influence of the main component “muscle strength” on the fall risk. In other words, factor analyzer 23 performs main component analysis based on the walking parameters to thus estimate a main component included in the factor of fall risk.

Factor analyzer 23 calculates the degree of influence for each of components 1 to 4 illustrated in FIG. 6, i.e. for each main component. The degree of influence may be an absolute value (for example, 6 points) based on scores, or a relative value (for example, 50%) based on the scores. In other words, in the case where the degree of influence is a value based on scores, factor analyzer 23 performs a process of calculating the degree of influence for each main component by combining (i.e. performing arithmetic operation on) the scores included in the fall risk value calculated by risk analyzer 22 for the main component.

Factor analyzer 23 then estimates a factor of fall risk of measured person 50 based on, for example, the degree of influence for each main component (S25). That is, factor analyzer 23 estimates the factor of fall risk based on two or more walking parameters. Based on the two or more walking parameters, factor analyzer 23 estimates one or more main components included in the factor of fall risk of measured person 50 from among the plurality of main components. As an example, factor analyzer 23 may estimate a main component whose degree of influence is highest, as the factor of fall risk of measured person 50. As another example, factor analyzer 23 may estimate a main component whose degree of influence is greater than or equal to a predetermined degree, as the factor of fall risk of measured person 50.

Following this, factor analyzer 23 outputs information indicating the estimation result to display device 40 (S26). That is, factor analyzer 23 causes display device 40 to display the estimation result.

Estimation device 20 then stores at least one of the moving image data, the walking parameters, or the estimation result in storage 24 (S27).

In the case where factor analyzer 23 obtains a determination result indicating that measured person 50 does not have fall risk from risk analyzer 22 (S23: No), factor analyzer 23 ends the process of estimating the factor of fall risk.

### [1-4. Effects, Etc.]

As described above, factor estimation system 1 according to this embodiment is a factor estimation system that estimates a factor of fall risk indicating a possibility of a fall of measured person 50, and includes: calculator 21 that obtains moving image data (an example of body motion data) indicating body motion of measured person 50 during walking, and calculates two or more walking parameters of measured person 50 based on the obtained body motion data; and factor analyzer 23 (an example of an estimator) that estimates, based on the two or more walking parameters, one or more main components that are included in the factor of the fall risk of measured person 50 and are based on the two or more walking parameters, and outputs an estimation result.

With this, factor analyzer 23 can estimate the factor of fall risk of measured person 50 based on two or more walking parameters. Specifically, factor analyzer 23 can estimate one or more main components based on two or more walking parameters. Factor estimation system 1 according to this embodiment can thus estimate the factor of fall risk.

Factor analyzer 23 estimates the one or more main components, based on information indicating a relationship between a physical strength index and the fall risk and information indicating a relationship between the physical strength index and the two or more walking parameters.

By using these information, factor analyzer 23 can estimate one or more main components from two or more walking parameters, with no need for measured person 50 to measure physical strength indexes. Hence, factor estimation system 1 can estimate the factor of fall risk more easily. In other words, factor analyzer 23 estimates the fall-related capacity of measured person 50 by using these information.

Factor estimation system 1 further includes risk analyzer 22 (an example of a second determiner) that determines the fall risk of measured person 50 based on the two or more walking parameters, and factor analyzer 23 estimates two or more main components when risk analyzer 22 determines that measured person 50 has the fall risk.

With this, factor estimation system 1 can determine the fall risk of measured person 50, such as whether measured person 50 has fall risk. The determination result, as a result of being output, can be notified to measured person 50 and/or his or her caregiver. Since the throughput of factor analyzer 23 can be reduced, factor estimation system 1 can save energy.

The one or more main components include at least one of muscle strength, muscle mass, balance, or cognitive function.

With this, when measured person 50 has fall risk, factor analyzer 23 can estimate whether the factor of fall risk is physical deterioration or cognitive deterioration.

The two or more walking parameters include at least two of walking speed, step length, joint angle, or lumbar displacement.

With this, factor analyzer 23 can estimate the factor of fall risk of measured person 50 based on at least two of walking speed, step length, joint angle, or lumbar displacement obtainable from the moving image data. That is, factor estimation system 1 can estimate the factor of fall risk of measured person 50 based on the moving image data obtained by capturing the usual walking state of measured person 50, without performing measurement for estimating the factor of fall risk (for example, measurement of physical strength indexes). Factor estimation system 1 can thus estimate the factor of fall risk more easily.

An estimation method in factor estimation system 1 according to this embodiment is a factor estimation method of estimating a factor of fall risk indicating a possibility of a fall of measured person 50, including: obtaining body motion data indicating body motion of measured person 50 during walking (S21); calculating two or more walking parameters of measured person 50 based on the body motion data obtained (S22); and estimating, based on the two or more walking parameters, one or more main components that are included in the factor of the fall risk of measured person 50 and are based on the two or more walking parameters (S25), and outputting an estimation result (S26).

This has the same effects as factor estimation system 1.

### Embodiment 2

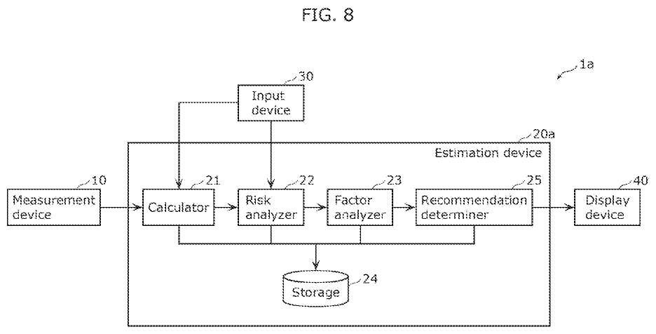

Factor estimation system 1a according to this embodiment will be described below, with reference to FIG. 8 to FIG. 13. Factor estimation system 1a according to this embodiment, in addition to estimating the factor of fall risk, suggests an intervention method for reducing the fall risk based on the estimation result.

The following will mainly describe the differences from Embodiment 1. The same structural elements as those in Embodiment 1 are given the same reference signs, and their description may be omitted or simplified.

### [2-1. Functional Structure of Factor Estimation System]

A functional structure of factor estimation system 1a according to this embodiment will be described below, with reference to FIG. 8. FIG. 8 is a block diagram illustrating the functional structure of factor estimation system 1a according to this embodiment.

As illustrated in FIG. 8, factor estimation system 1a includes estimation device 20a instead of estimation device 20 included in factor estimation system 1 according to Embodiment 1. Estimation device 20a includes recommendation determiner 25 in addition to the structure of estimation device 20 according to Embodiment 1.

Recommendation determiner 25 performs, based on the estimation result of the factor of fall risk of measured person 50, a process for enabling a caregiver or the like to intervene with measured person 50 according to the estimation result. For example, recommendation determiner 25 performs a process for suggesting a method with high intervention efficiency to the caregiver. Recommendation determiner 25 performs, for example, a determination process for suggesting the method (improvement menu) with high intervention efficiency to the caregiver, based on two or more main components included in the estimation result. The term “method with high intervention efficiency” denotes a method (improvement menu) that enables an intervention suitable for the factor of fall risk of measured person 50 to be made to measured person 50. That is, the method with high intervention efficiency is a method that can effectively reduce the fall risk of measured person 50. In detail, recommendation determiner 25 determines (decides) a reduction method for reducing the fall risk of measured person 50. Recommendation determiner 25 performs, for example, the determination according to the degree of influence of each of the two or more main components on the fall risk. Recommendation determiner 25 is an example of a first determiner.

For example, recommendation determiner 25 may store the determination result in storage 24, in order to analyze the long-term changes of the determination result. The determination result is an example of information based on body motion.

Recommendation determiner 25 may include a communication module (communication circuit) for performing wire communication or wireless communication. In this case, recommendation determiner 25 can use any communication method (communication standard, communication protocol) that enables communication with display device 40.

### [2-2. Operation in Factor Estimation System]

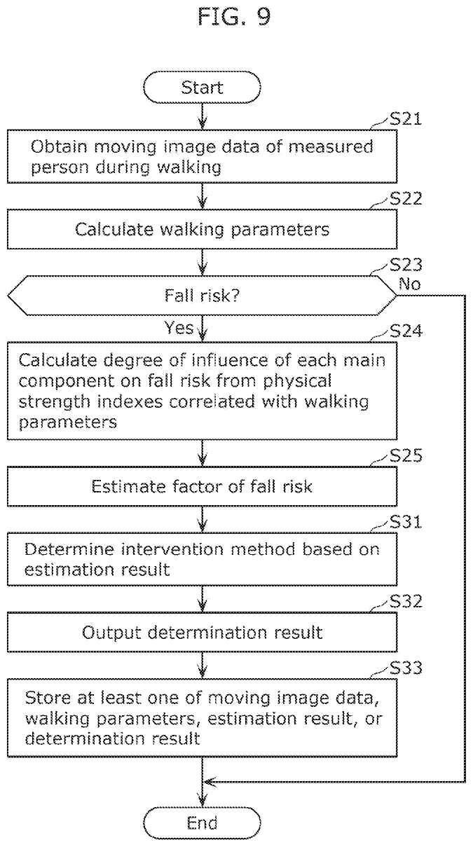

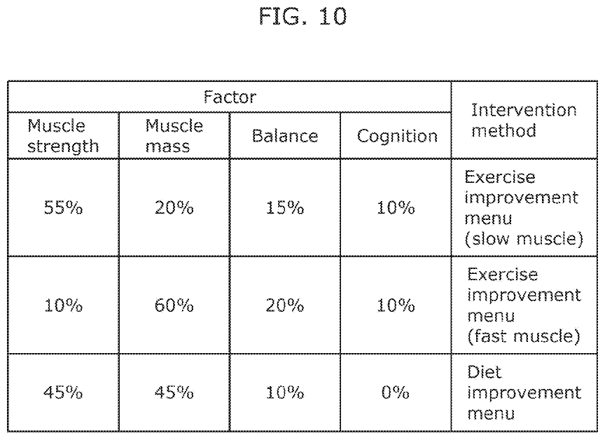

Operation in factor estimation system 1a according to this embodiment will be described below, with reference to FIG. 9 and FIG. 10. FIG. 9 is a flowchart illustrating operation in factor estimation system 1a according to this embodiment. Specifically, FIG. 9 illustrates operation of making a suggestion for reducing the fall risk based on the estimation result of the factor of fall risk of measured person 50. The processes in Steps S21 to S25 illustrated in FIG. 9 are the same as those illustrated in FIG. 7 in Embodiment 1, and their description is omitted.

As illustrated in FIG. 9, factor analyzer 23 estimates the factor of fall risk of measured person 50 (S25), and outputs the estimation result to recommendation determiner 25.

Having obtained the estimation result from factor analyzer 23, recommendation determiner 25 determines an intervention method to be recommended to, for example, the caregiver of measured person 50 based on the estimation result (S31). For example, recommendation determiner 25 determines an intervention method corresponding to the estimation result from among a plurality of intervention methods stored in storage 24. FIG. 10 is a diagram illustrating an example of the correspondence relationship between factors and intervention methods.

As illustrated in FIG. 10, recommendation determiner 25 determines an intervention method corresponding to the degrees of influence (proportions) of “muscle strength”, “muscle mass”, “balance”, and “cognition” on the fall risk. For example, in the case where the proportion of “muscle strength” is highest of “muscle strength”, “muscle mass”, “balance”, and “cognition”, recommendation determiner 25 determines “exercise improvement menu (slow muscle)” as a recommended intervention method. Developing the slow muscles improves the muscle strength effectively, so that the fall risk can be reduced easily.

For example, in the case where the proportion of “muscle mass” is highest of “muscle strength”, “muscle mass”, “balance”, and “cognition”, recommendation determiner 25 determines “exercise improvement menu (fast muscle)” as a recommended intervention method. Developing the fast muscles increases the muscle mass effectively, so that the fall risk can be reduced easily.

Thus, for example in the case where the proportion of one of “muscle strength” and “muscle mass” is highest, recommendation determiner 25 suggests an exercise menu for fall prevention and motor function improvement.

For example, in the case where the proportions of “muscle strength” and “muscle mass” from among “muscle strength”, “muscle mass”, “balance”, and “cognition” are similar (for example, equal), recommendation determiner 25 determines “diet improvement menu” as a recommended intervention method. Thus, for example in the case where the proportions of muscle-related main components such as “muscle strength” and “muscle mass” are similar, recommendation determiner 25 may suggest a method of improvement by diet instead of muscle training. The term “similar” may denote, for example, that the difference between the two proportions is less than or equal to a predetermined value. The predetermined value may be, for example, 10%, 20%, or the like.

The proportions illustrated in FIG. 10 are each calculated, for example, based on the sum of scores for the corresponding main component.

In FIG. 10, “cognition” denotes the degree of influence of cognitive function decline on walking. It is known that a person suffering from cognitive function decline or showing a sign of cognitive function decline and a person (healthy person) without cognitive function decline differ in body motion during walking. Hence, the degree of influence of “cognition” can be calculated, for example, based on walking parameters. The cognitive function denotes the ability of perception, memory, decision making, and the like.

An example of a cognitive function analysis method (evaluation method) will be described below, although the cognitive function analysis method is not limited to such.

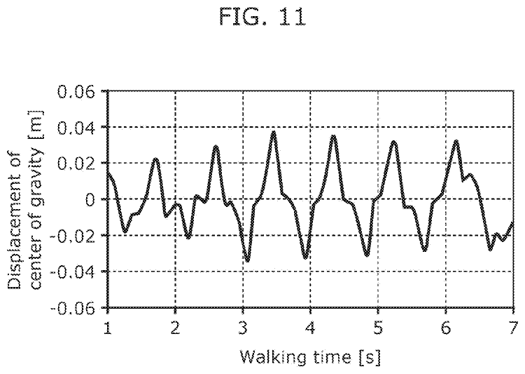

For example, in the case where the walking parameters include head position, factor analyzer 23 performs frequency analysis on head displacement. Factor analyzer 23 discrete Fourier transforms head displacement (for example, signal indicating the temporal change of the head position illustrated in FIG. 11). That is, factor analyzer 23 performs a frequency conversion process of converting a signal indicating body displacement from time domain to frequency domain. FIG. 11 is a diagram illustrating the vertical displacement of the body of measured person 50 during walking. The head displacement is an example of a change of the position of the center of gravity, and is, for example, calculated by calculator 21.

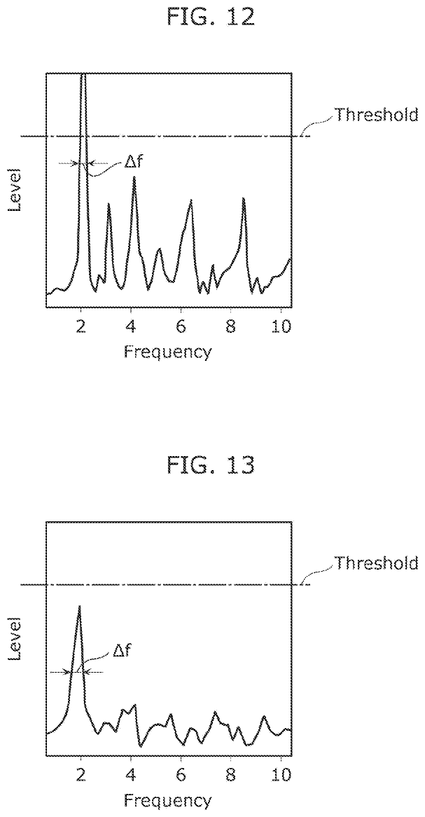

In the case where the cognitive function of measured person 50 is normal, an analysis result illustrated in FIG. 12 is obtained. In the case where the cognitive function of measured person 50 is low, an analysis result illustrated in FIG. 13s obtained. FIG. 12 is a diagram illustrating a frequency analysis result in the case where the cognitive function of measured person 50 is normal. FIG. 13 is a diagram illustrating a frequency analysis result in the case where the cognitive function of measured person 50 is low.

In each of the analysis results illustrated in FIG. 12 and FIG. 13, the peak of the lowest frequency (i.e. the peak of the highest level) is a frequency peak indicating the walking cycle. In other words, the peak of the lowest frequency is a main frequency component. If the cognitive function of measured person 50 is normal, measured person 50 can walk in a constant cycle. Accordingly, the frequency peak indicating the walking cycle in FIG. 12 is sharper and has the higher peak level than in FIG. 13.

If the cognitive function of measured person 50 is low, on the other hand, measured person 50 has difficulty in walking in a constant cycle, so that the walking cycle varies more. Accordingly, the frequency peak indicating the walking cycle in FIG. 13 has the lower peak level and is gentler than in FIG. 12.

Hence, factor analyzer 23 analyzes the cognitive function of measured person 50 based on the frequency peak indicating the walking cycle of measured person 50, which is obtained as a result of frequency analysis by discrete Fourier transform. For example, factor analyzer 23 analyzes the cognitive function of measured person 50 based on the peak level of the frequency peak. Factor analyzer 23 analyzes that the cognitive function of measured person 50 is lower when the peak level is lower. For example, in the case where the peak level is greater than or equal to a threshold (see FIG. 12 and FIG. 13), factor analyzer 23 determines that the cognitive function is normal, and assigns a score indicating that the cognitive function is normal. The score indicating that the cognitive function is normal is set beforehand, and may be, for example, 0 points.

For example, in the case where the peak level is less than the threshold, factor analyzer 23 determines that the cognitive function is low, and assigns a score indicating that the cognitive function is low. The score indicating that the cognitive function is low is set beforehand, and may be, for example, 2 points. The threshold may be, for example, stored in storage 24.

Referring back to FIG. 9, recommendation determiner 25 outputs information indicating the determination result to display device 40 (S32). That is, recommendation determiner 25 causes display device 40 to display the determination result. The determination result output from recommendation determiner 25 is an example of a first determination result.

Estimation device 20 then stores at least one of the moving image data, the walking parameters, the estimation result, or the determination result in storage 24 (S33). Recommendation determiner 25 may store the determination result in storage 24.

### [2-3. Effects, Etc.]

As described above, factor analyzer 23 in factor estimation system 1a according to this embodiment estimates two or more main components. Factor estimation system 1a further includes recommendation determiner 25 (an example of a first determiner) that determines, based on the two or more main components, an intervention method for measured person 50 to reduce the fall risk, and outputs a determination result.

With this, recommendation determiner 25 can suggest an intervention method suitable for the factor of fall risk to the caregiver or the like. Even in the case where the caregiver or the like does not have knowledge for fall risk reduction, factor estimation system 1a can urge the caregiver to reduce the fall risk of measured person 50 by an appropriate intervention method.

Recommendation determiner 25 determines an intervention method for reducing the degree of influence of a main component whose degree of influence on the fall risk is highest of the two or more main components.

With this, recommendation determiner 25 can output an intervention method that can effectively reduce the fall risk of measured person 50.

### Embodiment 3

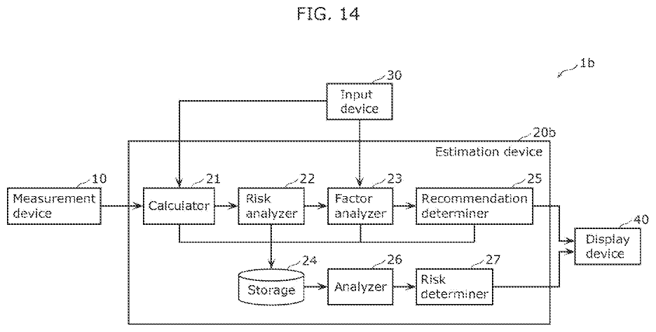

Factor estimation system 1b according to this embodiment will be described below, with reference to FIG. 14 to FIG. 16B. Factor estimation system 1b according to this embodiment has a feature that a fall risk-related process is performed based on past time-series data. Herein, “past time-series data” is time-series data obtained in the past. Examples include time-series data of the most recent one week, time-series data of the most recent one month, and time-series data of the most recent one year.

The following will mainly describe the differences from Embodiment 2. The same structural elements as those in Embodiment 2 are given the same reference signs, and their description may be omitted or simplified.

### [3-1. Functional Structure of Factor Estimation System]

A functional structure of factor estimation system 1b according to this embodiment will be described below, with reference to FIG. 14. FIG. 14 is a block diagram illustrating the functional structure of factor estimation system 1b according to this embodiment.

As illustrated in FIG. 14, factor estimation system 1b includes estimation device 20b instead of estimation device 20a included in factor estimation system 1a according to Embodiment 2. Estimation device 20b includes analyzer 26 and risk determiner 27 in addition to the structure of estimation device 20a according to Embodiment 2. Recommendation determiner 25 in estimation device 20b may be omitted.

Analyzer 26 analyzes information based on past body motion during walking. For example, analyzer 26 obtains the temporal change of fall risk-related time-series data by statistical processing. Analyzer 26 may then calculate the tendency of the time-series data or calculate a threshold for determining the current fall risk, by analyzing the temporal change of the time-series data. For example, analyzer 26 may analyze the temporal change of the past fall risk value of the user to calculate a threshold for the fall risk value of the user.

An example in which analyzer 26 analyzes fall risk-related time-series data will be described below, although the data to be analyzed is not limited to such and may be at least one of time-series data of the walking parameters, time-series data of the main component (for example, muscle strength) of the factor, or time-series data of the determination result. For example, as a result of analyzer 26 analyzing time-series data of the main component of the factor, risk determiner 27 can determine whether the degree of influence of the muscle strength on the fall risk is reduced, that is, whether the fall risk is reduced by the menu based on the intervention method.

Risk determiner 27 determines the fall risk of measured person 50 based on the analysis result of analyzer 26. Risk determiner 27 determines the fall risk of measured person 50 based on time-series data of at least one type of information based on past body motion during walking. For example, in the case where analyzer 26 calculates a threshold for the fall risk value of measured person 50, risk determiner 27 may determine whether the user has fall risk depending on whether the current fall risk is greater than the threshold. Thus, analyzer 26 may use past time-series data in order to set a threshold for information based on the current body motion during walking.

With this, for example in the case where the fall risk value of measured person 50 increases suddenly, risk determiner 27 can make determination depending on the increase.

Risk determiner 27 may include a communication module (communication circuit) for performing wire communication or wireless communication. In this case, risk determiner 27 can use any communication method (communication standard, communication protocol) that enables communication with display device 40.

### [3-2. Operation in Factor Estimation System]

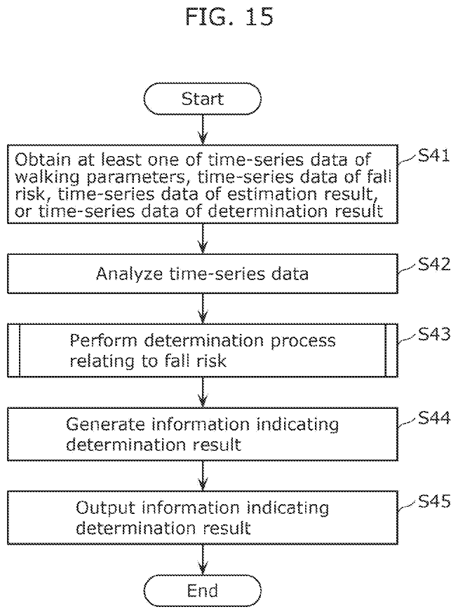

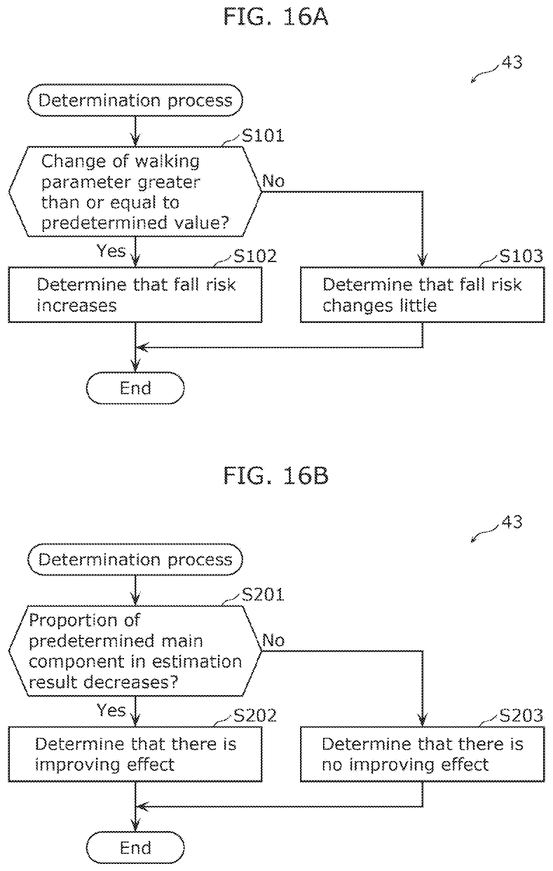

Operation in factor estimation system 1b according to this embodiment will be described below, with reference to FIG. 15 to FIG. 16B, FIG. 15 is a flowchart illustrating operation in factor estimation system 1b according to this embodiment.

As illustrated in FIG. 15, analyzer 26 obtains at least one of time-series data of the walking parameters, time-series data of the fall risk, time-series data of the estimation result, or time-series data of the determination result (S41). For example, analyzer 26 obtains the time-series data by reading the time-series data from storage 24.

Analyzer 26 then analyzes the time-series data (S42). For example, in the case where analyzer 26 obtains the walking parameters including walking speed in Step S41, analyzer 26 may calculate information indicating the degree of change in the walking speed relative to walking speed at a predetermined time. The degree of change may be, for example, the difference or ratio between the walking speed at the predetermined time and the walking speed at other than the predetermined time.

For example, in the case where analyzer 26 obtains the fall risk including a fall risk value in Step S41, analyzer 26 may calculate information indicating the degree of change in the fall risk value relative to a fall risk value at a predetermined time. The degree of change may be, for example, the difference or ratio between the fall risk value at the predetermined time and the fall risk value at other than the predetermined time.

For example, in the case where analyzer 26 obtains the degree of influence (for example, proportion illustrated in FIG. 10) of each main component in Step S41, analyzer 26 may calculate the tendency of the change in the proportion of the main component. For example, analyzer 26 may generate a line graph indicating the tendency.

For example, in the case where analyzer 26 obtains the determination result including an intervention method in Step S41, analyzer 26 may calculate the tendency of the change in the intervention method. For example, analyzer 26 may calculate, for each of a plurality of intervention methods, the number of times the intervention method was suggested in a predetermined period.

Analyzer 26 may perform statistical processing for the foregoing numeric value (for example, the degree of change, the fall risk value, the degree of influence, or the number of times). The statistics calculated in the statistical processing are, for example, mean values, but may also be maximum values, minimum values, median values, numeric values indicating variation (for example, standard deviations), and the like.

Analyzer 26 outputs the analysis result to risk determiner 27.

Risk determiner 27 performs a determination process relating to fall risk, based on the analysis result (S43). In other words, risk determiner 27 performs the determination process relating to fall risk based on the time-series data. For example, risk determiner 27 performs at least one of a determination process illustrated in FIG. 16A or a determination process illustrated in FIG. 16B. FIG. 16A is a flowchart illustrating an example of operation in risk determiner 27 according to this embodiment. FIG. 16A illustrates a flowchart in the case where the time-series data of the walking parameters is obtained in Step S41.

As illustrated in FIG. 16A, risk determiner 27 determines whether the change of any of the walking parameters is greater than or equal to a predetermined value (S101). For example, risk determiner 27 determines whether the change in walking speed is greater than or equal to a predetermined value. Risk determiner 27 determines, for example, whether the amount of decrease in walking speed is greater than or equal to the predetermined value.

In the case where the change of the walking parameter is greater than or equal to the predetermined value (S101: Yes), risk determiner 27 determines that the fall risk increases (S102). In the case where the change of the walking parameter is less than the predetermined value (S101: No), risk determiner 27 determines that the fall risk changes little (S103). For example, in the case where the change of the walking parameter is a predetermined change, risk determiner 27 may determine that the fall risk decreases. An example of the predetermined change is such a change that approaches an optimum value of the walking parameter.

In this way, factor estimation system 1b can notify the caregiver or the like of the tendency of the change of the fall risk of measured person 50.

Another example of operation in risk determiner 27 will be described below, with reference to FIG. 16B. FIG. 16B is a flowchart illustrating another example of operation in risk determiner 27 according to this embodiment. FIG. 16B illustrates a flowchart in the case where the time-series data of the estimation result is obtained in Step S41.

As illustrated in FIG. 16B, risk determiner 27 determines whether the proportion of a predetermined main component in the estimation result decreases (S201). For example, risk determiner 27 determines whether the proportion of “muscle strength” in the estimation result decreases. An example of the predetermined main component is a main component whose degree of influence on the fall risk was highest of the plurality of main components at least once in a predetermined period.

In the case where the proportion of the predetermined main component decreases (S201: Yes), risk determiner 27 determines that there is an effect of improvement by the intervention method (S202). In the case where the proportion of the predetermined main component does not decrease (S201: No), risk determiner 27 determines that there is no effect of improvement by the intervention method (S203). The determination by risk determiner 27 in Step S201 may result in Yes in the case where the decrease of the proportion of the predetermined main component is a predetermined degree or more.

Referring back to FIG. 15, risk determiner 27 then generates information indicating the determination result (S44), and outputs the generated information indicating the determination result to display device 40 (S45). That is, risk determiner 27 causes display device 40 to display the determination result.

In this way, factor estimation system 1b can notify the caregiver or the like of the effect of improvement by the intervention method.

The timing at which factor estimation system 1 performs the foregoing operation is not limited, and the operation may be performed periodically.

### [3-3. Effects, Etc.]

As described above, factor estimation system 1b according to this embodiment further includes risk determiner 27 (an example of a third determiner) that determines the fall risk based on at least one of time-series data of the two or more walking parameters, time-series data of the estimation result, or time-series data of the determination result of risk analyzer 22.

With this, risk determiner 27 can determine the fall risk based on the temporal changes in the two or more walking parameters, the estimation result, and/or the determination result of risk analyzer 22, so that the fall risk can be detected early.

### OTHER EMBODIMENTS

While each embodiment (hereafter also referred to as “embodiments, etc.”) has been described above, the present disclosure is not limited to the foregoing embodiments, etc.

For example, although the foregoing embodiments, etc, describe an example in which the estimation device does not include the measurement device, the input device, and the display device, i.e. the estimation device is separate from the measurement device, the input device, and the display device, the present disclosure is not limited to such. The estimation device may have the function of at least one of the measurement device, the input device, or the display device. In this case, the measurement device functions as a measurer in the estimation device, the input device functions as an inputter in the estimation device, and/or the display device functions as a display in the estimation device. For example, the factor estimation system may be composed of one device.

Although the foregoing embodiments, etc. describe an example in which the estimation device in the factor estimation system is implemented by a single device, the estimation device may be implemented by a plurality of devices. For example, the estimation device may be implemented by one server device, or three or more server devices. In the case where the factor estimation system is implemented by a plurality of server devices, the structural elements in the estimation device may be allocated to the plurality of server devices in any way.

Each of the structural elements in the foregoing embodiments, etc. may be configured in the form of an exclusive hardware product, or may be implemented by executing a software program suitable for the structural element. Each of the structural elements may be implemented by means of a program executing unit, such as a CPU or a processor, reading and executing the software program recorded on a recording medium such as a hard disk or a semiconductor memory.

The processes performed by any specific processing unit in the foregoing embodiments, etc. may be performed by another processing unit. The orders of processes described in the flowcharts in the foregoing embodiments, etc, are merely examples. A plurality of processes may be changed in order, and a plurality of processes may be performed in parallel.

Each of the structural elements in the foregoing embodiments, etc. may be implemented by executing a software program suitable for the structural element. Each of the structural elements may be implemented by means of a program executing unit, such as a CPU or a processor, reading and executing the software program recorded on a recording medium such as a hard disk or a semiconductor memory.

Each of the structural elements may be implemented by hardware. For example, the structural elements may be circuits (or integrated circuits). These circuits may constitute one circuit as a whole, or may be separate circuits. These circuits may each be a general-purpose circuit or a dedicated circuit.

The division of the functional blocks in each block diagram is an example, and a plurality of functional blocks may be realized as one functional block, one functional block may be divided into a plurality of functional blocks, or part of functions may be transferred to another functional block. Moreover, functions of a plurality of functional blocks having similar functions may be realized by single hardware or software in parallel or in a time-sharing manner.

The general and specific aspects of the present disclosure may be implemented using a system, a method, an integrated circuit, a computer program, or a computer-readable recording medium such as CD-ROM, or any combination of systems, methods, integrated circuits, computer programs, and recording media.

Other modifications obtained by applying various changes conceivable by a person skilled in the art to each embodiment of the structural elements and functions in each embodiment without departing from the scope of the present disclosure are also included in the present disclosure.

### REFERENCE SIGNS LIST

1, 1a, 1b factor estimation system
21 calculator
22 risk analyzer (second determiner)
23 factor analyzer (estimator)
recommendation determiner (first determiner)
27 risk determiner (third determiner)
50 measured person
D1 first correspondence information
D2 second correspondence information

1. A factor estimation system that estimates a factor of fall risk indicating a possibility of a fall of a measured person, the factor estimation system comprising:
a calculator that obtains body motion data indicating body motion of the measured person during walking, and calculates two or more walking parameters of the measured person based on the body motion data obtained; and
an estimator that estimates, based on the two or more walking parameters, one or more main components that are included in the factor of the fall risk of the measured person and are based on the two or more walking parameters, and outputs an estimation result.

2. The factor estimation system according to claim 1,
wherein the estimator estimates the one or more main components, based on information indicating a relationship between a physical strength index and the fall risk and information indicating a relationship between the physical strength index and the two or more walking parameters.

3. The factor estimation system according to claim 1,
wherein the one or more main components estimated by the estimator are two or more main components, and
the factor estimation system further comprises:
a first determiner that determines, based on the two or more main components, an intervention method for the measured person to reduce the fall risk, and outputs a determination result.

4. The factor estimation system according to claim 3,
wherein the first determiner determines the intervention method for reducing a degree of influence of a main component whose degree of influence on the fall risk is highest of the two or more main components.

5. The factor estimation system according to claim 3, further comprising:
a second determiner that determines the fall risk of the measured person based on the two or more walking parameters,
wherein the estimator estimates the two or more main components, when the second determiner determines that the measured person has the fall risk.

6. The factor estimation system according to claim 5, further comprising:
a third determiner that determines the fall risk based on at least one of time-series data of the two or more walking parameters, time-series data of the estimation result, or time-series data of a determination result of the second determiner.

7. The factor estimation system according to claim 1,
wherein the one or more main components include at least one of muscle strength, muscle mass, balance, or cognitive function.

8. The factor estimation system according to claim 1,
wherein the two or more walking parameters include at least two of walking speed, step length, joint angle, or lumbar displacement.

9. A factor estimation method of estimating a factor of fall risk indicating a possibility of a fall of a measured person, the factor estimation method comprising:
obtaining body motion data indicating body motion of the measured person during walking;
calculating two or more walking parameters of the measured person based on the body motion data obtained; and
estimating, based on the two or more walking parameters, one or more main components that are included in the factor of the fall risk of the measured person and are based on the two or more walking parameters, and outputting an estimation result.
